(19) United States
(12) Patent Application Publication
(10) Pub. No.: US 2019/0022628 A1
(43) Pub. Date: Jan. 24, 2019

(54) POST IMPREGNATION HEAT TREATMENT FOR SILVER-BASED EPOXIDATION CATALYSTS

(71) Applicant: Scientific Design Company, Inc., Little Ferry, NJ (US)

(72) Inventors: Serguei PAK, Teaneck, NJ (US); Andrew MCFARLAND, South Hackensack, NJ (US)

(73) Assignee: Scientific Design Company, Inc., Little Ferry, NJ (US)

(21) Appl. No.: 16/043,296

(22) Filed: Jul. 24, 2018

Related U.S. Application Data
(60) Provisional application No. 62/536,138, filed on Jul. 24, 2017.

(51) Int. Cl. B01J 23/68; B01J 21/04; B01J 35/00; B01J 37/02; C07D 301/03

(52) U.S. Cl. CPC B01J 23/688; B01J 21/04; C07D 301/03; B01J 37/0201; B01J 35/0006

(57) ABSTRACT

The present disclosure is directed to the preparation of silver-based HSCs. During preparation of the catalyst a selected carrier is co-impregnated with a solution containing a catalytically effective amount of silver and a promoting amount of rhenium and other promoters. After co-impregnation, the carrier is subjected to a separate heat treatment prior to calcination. Such heat treatment is conducted for between about 1 minute and about 120 minutes at temperatures between about 40° C. and about 300° C. Catalysts prepared by the present methodology evidence improved selectivity, activity and/or stability resulting in an increase in the useful life of the catalyst.

### CROSS-REFERENCE TO RELATED APPLICATIONS

This application claims the benefit of U.S. Provisional Application No. 62/536,138 filed on Jul. 24, 2017, the entire contents of which are incorporated herein by reference.

### FIELD OF THE INVENTION

The present disclosure relates to silver-based ethylene oxide catalysts for the oxidative conversion of ethylene to ethylene oxide, and particularly to the preparation of such catalysts. More specifically, the present disclosure is directed to a method of producing such silver-based catalysts exhibiting improved activity, selectivity and/or stability by virtue of the present methodology of catalyst preparation. This method particularly employs a heat treatment step after co-impregnation, and before calcination of the catalyst during catalyst preparation.

### BACKGROUND

As known in the art, high selectivity catalysts (HSCs) for the epoxidation of ethylene refer to those catalysts that possess selectivity values higher than high activity catalysts (HACs) used for the same purpose. Both types of catalysts include silver as the active catalytic component on a refractory support (i.e., “carrier”, such as alumina). Typically, one or more promoters are included in the catalyst to improve or modify properties of the catalyst, such as selectivity.

Generally, HSCs achieve the higher selectivity (typically, in excess of 87 mole %) by incorporation of rhenium as a promoter. Typically, one or more additional promoters selected from alkali metals (e.g., cesium), alkaline earth metals (e.g., strontium), transition metals (e.g., tungsten compounds), and main group elements (e.g., sulfur and/or halide compounds) are also included.

Nevertheless, there remains a need to improve the activity and selectivity performance of HSCs. Moreover, it is well known that with use of a catalyst, the catalyst will age (i.e., degrade) until use of the catalyst is no longer practical, i.e., when activity and selectivity values diminish to a level that is no longer industrially efficient or economical. Thus, there is a further continuous need to extend the useful lifetime (i.e., “longevity” or “usable life”) of these catalysts by maintaining an effective level of activity and selectivity characteristics. The useful lifetime of the catalyst is directly dependent on the stability of the catalyst. As used herein, the “useful lifetime” is the time period for which a catalyst can be used until one or more functional parameters, such as selectivity or activity, degrade to such a level that use of the catalyst becomes impractical. Although many approaches for boosting the activity, selectivity, and/or stability of the catalyst have been undertaken, there remains a need for further improvements and a more straight-forward and cost-effective method for achieving such an improved catalyst.

### SUMMARY

The present method is directed to the preparation of silver-based HSCs effective for the conversion of ethylene to ethylene oxide. Specifically, the method includes co-impregnating a porous refractory alumina carrier with a solution containing a catalytically effective amount of silver and a promoting amount of rhenium and other promoters, wherein after the co-impregnation is complete, the impregnated alumina carrier is heated at a temperature of about 40° C. to about 300° C. for a duration of about 1 minute to about 120 minutes prior to calcination. Thereafter the heat treated co-impregnated carrier is calcined for a time and at a temperature sufficient to convert the contained silver to an active species. Surprisingly, catalysts prepared in accordance with the present method exhibit enhanced performance, including improved selectivity, activity and/or stability relative to catalysts prepared in the absence of the identified heat treatment step. Such treatment ultimately extends the useful life of the HSC.

### DETAILED DESCRIPTION

The present disclosure is directed to a method for the preparation of a silver-based HSC which improves the performance, i.e., activity, selectivity and/or stability, of the catalyst compared with conventionally prepared silver-based HSCs reflected in the art. Specifically, the present method includes the heat treatment of a co-impregnated refractory carrier prior to conventional calcination during the preparation of the HSC. More specifically, a selected carrier is impregnated with a catalytically effective amount of silver and coincidentally co-impregnated with selected promoters including promoting amounts of one or more of rhenium, alkali metals and alkali earth metals, for example. After completion of the co-impregnation of the carrier by conventional methods, the carrier is subjected to a separate heat treatment, during which the co-impregnated carrier is heated to a temperature of about 40° C. to about 300° C. for a duration of between 1 minute and about 120 minutes. After the heat treatment is completed, the carrier is calcined for a time sufficient to remove the volatile components from the co-impregnated, heat treated support and to convert the remaining silver containing compound to an active silver species. The carrier treated in accordance with the present method, particularly characterized by heat treatment, provides a catalyst which exhibits improved selectivity, activity and/or stability and improves the useful life of the catalyst.

While not wishing to be bound, it is understood that silver-based HSC performance is improved by post-impregnation heat treatment when conducted at conditions initiating, for example, Ag-amine complex decomposition and preferential deposition of Ag on and into the carrier. This is particularly effective while the impregnation solution evidences little or no evaporation and the impregnation solution maintains the solubility of the promoters, i.e., in an ion soluble solution. As a consequence of the heat treatment of the co-impregnated carrier, higher loss of Ag concentration is observed at or near the surface of the carrier (as measured by XPS analysis) resulting from a higher incidence of promoter deposition on the silver particle, creating more catalytically active sites, and thus leading to the improvements exhibited by the heat-treated HSC.

The support (i.e., carrier) may comprise materials such as alpha-alumina, charcoal, pumice, magnesia, zirconia, Mania, kieselguhr, fuller's earth, silicon carbide, silica, silicon carbide, clays, artificial zeolites, natural zeolites, silicon dioxide and/or titanium dioxide, ceramics and combination thereof. The preferred support is comprised of alpha-alumina having a very high purity; i.e., at least 95 wt. % pure, or more preferably, at least 98 wt. % alpha-alumina. The remaining components may include inorganic oxides other than alpha-alumina, such as silica, alkali metal oxides (e.g., sodium oxide) and trace amounts of other metal-containing or non-metal-containing additives or impurities.

The carrier can have any suitable distribution of pore diameters. As used herein, the term “pore diameter” is meant to indicate a pore size. The pore volume (and pore size distribution) described herein can be measured by any suitable method, such as by the conventional mercury porosimeter method described in, for example, Drake and Ritter, Ind. Eng. Chem. Anal. Ed., 17, 787 (1945). Typically, the pore diameters are at least about 0.01 microns (0.01 μm), and more typically, at least about 0.1 μm. Typically, the pore diameters are no more than or less than about 10, 15, 20, 25, 30, 35, 40, 45, or 50 μm. In different embodiments, the pore diameters are about, at least, above, up to, or less than, for example, 0.2 μm, 0.5 μm, 1.0 μm, 1.2 μm, 1.5 μm, 1.8 μm, 2.0 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm, or 10.5 μm, or the pore diameters are within a range bounded by any two of the foregoing exemplary values. Any range of pore sizes, as particularly derived from any of the above exemplary values, may also contribute any suitable percentage of the total pore volume, such as at least, greater than, up to, or less than, for example, 1, 2, 5, 10, 15, 20, 25, 30, 40, 50, 60, 70, 80, 90, 95, or 98% of the total pore volume. In some embodiments, a range of pore sizes may provide the total (i.e., 100%) pore volume.

The final support typically, but not necessarily always, has a water absorption value ranging from about 0.2 cc/g to about 0.8 cc/g, preferably from about 0.25 cc/g to about 0.6 cc/g. The BET surface area of the finished support is preferred to be in the range from about 0.3 to about 4.0 m²/g, more preferably from about 0.3 to about 1.5 m²/g, and most preferably from about 0.3 m²/g to about 1 m²/g. Suitable porosity volumes measured by mercury intrusion techniques are generally in the range from about 0.2 mug to about 0.8 ml/g, and preferably from about 0.25 ml/g to about 0.60 ml/g.

Regardless of the character of the support used, it is usually shaped into particles, chunks, pieces, pellets, rings, spheres, wagon wheels, cross-partitioned hollow cylinders, and the like, of a size suitable for employment in a fixed-bed epoxidation reactor. The type of reactor is not limited as long as it is capable of producing an olefin oxide by the catalytic oxidation of an olefin. Desirably, the support particles may have equivalent diameters in the range from about 3 mm to about 12 mm, and preferably in the range from about 5 mm to about 10 mm, which are usually compatible with the internal diameter of the tubular reactors in which the catalyst is placed. Equivalent diameter is the diameter of a sphere having the same external surface (i.e., neglecting surface within the pores of the particle) to volume ratio as the support particles being employed.

In general and as briefly mentioned above, a suitable catalyst support of the present invention can be prepared by mixing the refractory material, such as alumina, water or other suitable liquid, a burnout material or suitable porosity-controlling agent, and a binder. Burnout materials include cellulose, substituted celluloses, e.g., methylcellulose, ethylcellulose, and carboxyethylcellulose, stearates, such as organic stearate esters, e.g., methyl or ethyl stearate, waxes, granulated polyolefins, particularly polyethylene and polypropylene, walnut shell flour, and the like which are decomposable at the firing temperatures used in preparation of the support. The burnout material is used to modify the porosity of the support and it is essentially totally removed during the firing to produce the finished support. Supports of the present invention are preferably made with the inclusion of a bonding material such as silica with an alkali metal compound in sufficient amount to substantially prevent the formation of crystalline silica compounds. Appropriate binders include inorganic clay-type materials. For instant, a particularly convenient binder material is a mixture of boehmite, an ammonia stabilized silica sol, and a soluble sodium salt.

A paste is formed by mixing the dry ingredients of the support with water or another suitable liquid, and the paste is usually extruded or molded into the desired shape, and then fired or calcined at a temperature from about 1200° C. to about 1600° C. to form the support. When the particles are formed by extrusion, it may be desirable to also include extrusion aids. The amounts of extrusion aids required would depend on a number of factors that relate to the equipment used. However these matters are well within the general knowledge of a person skilled in the art of extruding ceramic materials. After firing, the support is preferably washed to remove soluble residues and/or to modify the surface structure and roughness. Washing is most commonly done with water, but washing with other solvents or aqueous/non-aqueous solutions can also be beneficial.

Silver-based epoxidation catalysts for the oxidation of an olefin to an olefin oxide are formed by providing a catalytically effective amount of silver on its surface. The catalyst is prepared by impregnating the support with a silver compound, complex or salt dissolved in a suitable solvent sufficient to cause deposition of a silver-precursor compound onto the support. Preferably, an aqueous silver solution is used.

Preferred catalysts prepared in accordance with this invention contain up to about 45% by weight of silver, expressed as metal, based on the total weight of the catalyst including the support. The silver is deposited upon the surface and throughout the pores of a porous refractory support. Silver contents, expressed as metal, from about 1% to about 40% based on the total weight of the catalyst are preferred, while silver contents from about 8% to about 35% are more preferred. The amount of silver deposited on the support or present on the support is that amount which is a catalytically effective amount of silver, i.e., an amount which economically catalyzes the reaction of ethylene and oxygen to produce ethylene oxide. As used herein, the term “catalytically effective amount of silver” refers to an amount of silver that provides a measurable conversion of ethylene and oxygen to ethylene oxide. Useful silver containing compounds which are silver precursors non-exclusively include silver oxalate, silver nitrate, silver oxide, silver carbonate, a silver carboxylate, silver citrate, silver phthalate, silver lactate, silver propionate, silver butyrate and higher fatty acid salts and combinations thereof.

Also deposited on the support, coincidentally with the deposition of the silver, in accordance with the present invention, is a promoting amount of a rhenium component, which may be a rhenium-containing compound or a rhenium-containing complex. The rhenium promoter may be present in an amount from about 0.001 wt. % to about 1 wt. %, preferably from about 0.005 wt. % to about 0.5 wt. %, and more preferably from about 0.01 wt. % to about 0.1 wt. % based on the weight of the total catalyst including the support, expressed as the rhenium metal.

Also deposited on the support, coincidentally with the deposition of the silver and rhenium, in accordance with the present invention, are promoting amounts of an alkali metal or mixtures of two or more alkali metals, as well as optional promoting amounts of a Group IIA alkaline earth metal component or mixtures of two or more Group IIA alkaline earth metal components, and/or a transition metal component or mixtures of two or more transition metal components, all of which may be in the form of metal ions, metal compounds, metal complexes and/or metal salts dissolved in an appropriate solvent. The carrier is preferably co-impregnated, with the silver compounds and, at the same time with the various catalyst promoters. The particular combination of support, silver, alkali metal promoter(s), rhenium component, and optional additional promoter(s) of the instant invention will provide an improvement in one or more catalytic properties over the same combination of silver and support and none, or only one of the promoters.

As used herein the term “promoting amount” of a certain component of the catalyst refers to an amount of that component that works effectively to improve the catalytic performance of the catalyst when compared to a catalyst that does not contain that component. The exact concentrations employed, of course, will depend on, among other factors, the desired silver content, the nature of the support, the viscosity of the liquid, and solubility of the particular compound used to deliver the promoter into the impregnating solution. Examples of catalytic properties include, inter alia, operability (resistance to runaway), selectivity, activity, conversion, stability and yield. It is understood by one skilled in the art that one or more of the individual catalytic properties may be enhanced by the “promoting amount” while other catalytic properties may or may not be enhanced or may even be diminished. It is further understood that different catalytic properties may be enhanced at different operating conditions. For example, a catalyst having enhanced selectivity at one set of operating conditions may be operated at a different set of conditions wherein the improvement shows up in the activity rather than the selectivity. In the epoxidation process, it may be desirable to intentionally change the operating conditions to take advantage of certain catalytic properties even at the expense of other catalytic properties. The preferred operating conditions will depend upon, among other factors, feedstock costs, energy costs, by-product removal costs and the like.

Suitable alkali metal promoters may be selected from lithium, sodium, potassium, rubidium, cesium or combinations thereof, with cesium being preferred, and combinations of cesium with other alkali metals being especially preferred. The amount of alkali metal deposited or present on the support is to be a promoting amount. Preferably, the amount ranges from about 10 ppm to about 3000 ppm, more preferably from about 15 ppm to about 2000 ppm, and even more preferably from about 20 ppm to about 1500 ppm, and as especially preferred from about 50 ppm to about 1000 ppm by weight of the total catalyst, measured as the metal.

Suitable alkaline earth metal promoters comprise elements from Group HA of the Periodic Table of the Elements, which may be beryllium, magnesium, calcium, strontium, and barium or combinations thereof. Suitable transition metal promoters may comprise elements from Groups IVA, VA, VIA, VIIA and VIIIA of the Periodic Table of the Elements, and combinations thereof. Most preferably the transition metal comprises an element selected from Groups IVA, VA or VIA of the Periodic Table of the Elements. Preferred transition metals that can be present include molybdenum, tungsten, chromium, titanium, hafnium, zirconium, vanadium, tantalum, niobium, or combinations thereof.

The amount of alkaline earth metal promoter(s) and/or transition metal promoter(s) deposited on the support is a promoting amount. The transition metal promoter may typically be present in an amount from about 0.1 micromoles per gram to about 10 micromoles per gram, preferably from about 0.2 micromoles per gram to about 5 micromoles per gram, and more preferably from about 0.5 micromoles per gram to about 4 micromoles per gram of total catalyst, expressed as the metal. The catalyst may further comprise a promoting amount of one or more sulfur compounds, one or more phosphorus compounds, one or more boron compounds, one or more halogen-containing compounds, or combinations thereof.

The silver solution used to impregnate the support may also comprise an optional solvent or a complexing/solubilizing agent such as are known in the art. A wide variety of solvents or complexing/solubilizing agents may be employed to solubilize silver to the desired concentration in the impregnating medium. Useful complexing/solubilizing agents include amines, ammonia, oxalic acid, lactic acid and combinations thereof. Amines include an alkylene diamine having from 1 to 5 carbon atoms. In one preferred embodiment, the solution comprises an aqueous solution of silver oxalate and ethylene diamine. The complexing/solubilizing agent may be present in the impregnating solution in an amount from about 0.1 to about 5.0 moles per mole of silver, preferably from about 0.2 to about 4.0 moles, and more preferably from about 0.3 to about 3.0 moles for each mole of silver.

When a solvent is used, it may be an organic solvent or water, and may be polar or substantially or totally non-polar. In general, the solvent should have sufficient solvating power to solubilize the solution components. At the same time, it is preferred that the solvent be chosen to avoid having an undue influence on or interaction with the solvated promoters. Examples of organic solvents include, but are not limited to, alcohols, in particular alkanols; glycols, in particular alkyl glycols; ketones; aldehydes; amines; tetrahydrofuran; nitrobenzene; nitrotoluene; glymes, in particular glyme, diglyme and tetraglyme; and the like. Organic-based solvents which have 1 to about 8 carbon atoms per molecule are preferred. Mixtures of several organic solvents or mixtures of organic solvent(s) with water may be used, provided that such mixed solvents function as desired herein.

The concentration of silver in the impregnating solution is typically in the range from about 0.1% by weight up to the maximum solubility afforded by the particular solvent/solubilizing agent combination employed. It is generally very suitable to employ solutions containing from 0.5% to about 45% by weight of silver, with concentrations from 5 to 35% by weight of silver being preferred.

Impregnation of the selected support is achieved using any of the conventional methods; for example, excess solution impregnation, incipient wetness impregnation, spray coating, etc. Typically, the support material is placed in contact with the silver-containing solution until a sufficient amount of the solution is absorbed by the support. Preferably the quantity of the silver-containing solution used to impregnate the porous support is no more than is necessary to fill the pores of the support.

After co-impregnation is complete, i.e., deposition with a silver-containing compound (a silver precursor) and one or more of a rhenium component, an alkali metal component and the optional other promoters, the co-impregnated carrier is subjected to a heat treatment step. Specifically, the co-impregnated carrier is heated for between about 1 minute and 120 minutes at a temperature from between about 40° C. and about 300° C. Preferably, the co-impregnated carrier is heated for about 10 minutes to about 60 minutes at a temperature between about 50° C. and about 200° C., and more preferably for between about 20 minutes and about 30 minutes at a temperature of between about 60° C. and about 100° C. The co-impregnated carrier can be heated, for example, at 80° C. for 30 minutes to achieve effective results, including, improvement in activity, selectivity and/or stability of the finished catalyst. Heating can be conducted preferably in air or an oxygen atmosphere but can be conducted in any atmosphere which does not affect the impregnation solution.

While not wishing to be bound, the heat treatment step is conducted at conditions initiating, for example, Ag-amine complex decomposition and the preferential deposition of Ag on the surface and into the carrier. This is particularly the case when the co-impregnation solution evidences little or no evaporation and the co-impregnation solution maintains the solubility of the promoters, i.e., in an ion soluble solution. HSC performance improves when the co-impregnated solution contains thermally unstable Ag complex and soluble and stable promoters. Notably, impregnation solution losses during heat treatment are preferred to be less than 50% by weight, more preferably less than 25% by weight, and even more preferably less than 10% by weight. By thermally unstable it is meant that the compounds or complexes are able to decompose at process or ambient temperatures.

As a consequence of the heat treatment of the co-impregnated support, higher deposition levels of promoters are observed on the deposited Ag particles indicating higher corresponding concentrations of active sites formed on the Ag particles. Notably, a higher loss of Ag concentration is observed (as measured by XPS analysis) at or near the surface of the heat treated co-impregnated support resulting from a higher incidence of promoter deposition on the Ag particles, creating a larger population of catalytically active sites. Notably, in this context, Ag surface coverage by promoters, measured as a loss of near surface atomic concentration of Ag (in XPS analysis) is preferably, more than 10%, more preferably, more than 20% and even more preferably, more that 30%.

After heat treatment, the co-impregnated support is calcined for a time sufficient to remove the volatile components from the impregnated support to result in a catalyst precursor and to convert the silver containing compound to an active silver species. The calcination may be accomplished by heating the impregnated support, preferably at a gradual rate, to a temperature in the range from about 200° C. to about 600° C., preferably from about 200° C. to about 500° C., and more preferably from about 200° C. to about 450° C., at a pressure in the range from about 0.5 to about 35 bar. In general, the higher the temperature, the shorter the required heating period. A wide range of heating periods have been suggested in the art; e.g., U.S. Pat. No. 3,563,914 discloses heating for less than 300 seconds, and U.S. Pat. No. 3,702,259 discloses heating from 2 to 8 hours at a temperature of from 100° C. to 375° C., usually for duration of from about 0.5 to about 8 hours. However, it is only important that the heating time be correlated with the temperature such that substantially all of the contained silver is converted to the active silver species. Continuous or step-wise heating may be used for this purpose.

During calcination, the impregnated support may be exposed to a gas atmosphere comprising an inert gas or a mixture of an inert gas with from about 10 ppm to 21% by volume of an oxygen-containing oxidizing component. For purposes of this invention, an inert gas is defined as a gas that does not substantially react with the catalyst or catalyst precursor under the conditions chosen for the calcination. Non-limiting examples include nitrogen, argon, krypton, helium, and combinations thereof, with the preferred inert gas being nitrogen. Non-limiting examples of the oxygen-containing oxidizing component include molecular oxygen (O₂), CO₂, NO, NO₂, N₂O, N₂O₃, N₂O₄, or N₂O₅, or a substance capable of forming NO, NO₂, N₂O, N₂O₃, N₂O₄, or N₂O₅ under the calcination conditions, or combinations thereof, and optionally comprising SO₃, SO₂ or combinations thereof. Of these, molecular oxygen is a useful embodiment, and a combination of O₂ with NO or NO₂ is another useful embodiment. In a useful embodiment, the atmosphere comprises from about 10 ppm to about 1% by volume of an oxygen-containing oxidizing component. In another useful embodiment, the atmosphere comprises from about 50 ppm to about 500 ppm of an oxygen-containing oxidizing component. Calcination in air is also effective.

In another embodiment, the heat treated co-impregnated support, which has been calcined as disclosed above, may optionally thereafter be contacted with an atmosphere comprising a combination of oxygen and steam, which atmosphere is substantially absent of an olefin, and preferably, completely absent of an olefin. The atmosphere usually comprises from about 2% to about 15% steam by volume, preferably from about 2% to about 10% steam by volume, and more preferably from about 2% to about 8% steam by volume. The atmosphere usually comprises from about 0.5% to about 30% oxygen by volume, preferably from about 1% to about 21% oxygen by volume, and more preferably from about 5% to about 21% oxygen by volume. The balance of the gas atmosphere may be comprised of an inert gas. Non-limiting examples of the inert gas include nitrogen, argon, krypton, helium, and combinations thereof, with the preferred inert gas being nitrogen. The contacting is usually conducted at a temperature from about 200° C. or higher. In one embodiment the contacting is conducted at a temperature from about 200° C. to about 350° C. In another embodiment the contacting is conducted at a temperature from about 230° C. to about 300° C. In another embodiment the contacting is conducted at a temperature from about 250° C. to about 280° C. In another embodiment the contacting is conducted at a temperature from about 260° C. to about 280° C. Usually the contacting is conducted for from about 0.15 hour or more. In one embodiment, the contacting is conducted for from about 0.5 hour to about 200 hours. In another embodiment, the contacting is conducted for from about 3 hours to about 24 hours. In another embodiment, the contacting is conducted for from about 5 hours to about 15 hours.

Olefin Oxide Production

The epoxidation process may be carried out by continuously contacting an oxygen-containing gas with an olefin, which is preferably ethylene, in the presence of the catalyst produced by the invention. Oxygen may be supplied to the reaction in substantially pure molecular form or in a mixture such as air. Molecular oxygen employed as a reactant may be obtained from conventional sources. By way of example, reactant feed mixtures may contain from about 0.5% to about 45% ethylene and from about 3% to about 15% oxygen, with the balance comprising comparatively inert materials including such substances as carbon dioxide, water, inert gases, other hydrocarbons, and one or more reaction modifiers such as organic halides. Non-limiting examples of inert gases include nitrogen, argon, helium and mixtures thereof. Non-limiting examples of the other hydrocarbons include methane, ethane, propane and mixtures thereof. Carbon dioxide and water are byproducts of the epoxidation process as well as common contaminants in the feed gases. Both have adverse effects on the catalyst, so the concentrations of these components are usually kept at a minimum. Non-limiting examples of reaction moderators include organic halides such as C₁ to C₈ halohydrocarbons. Preferably, the reaction moderator is methyl chloride, ethyl chloride, ethylene dichloride, ethylene dibromide, vinyl chloride or mixtures thereof. Most preferred reaction moderators are ethyl chloride and ethylene dichloride. Usually such reaction moderators are employed in an amount from about 0.3 to about 20 ppmv, and preferably from about 1 to about 15 ppmv of the total volume of the feed gas.

A usual method for the ethylene epoxidation process comprises the vapor-phase oxidation of ethylene with molecular oxygen, in the presence of the inventive catalyst, in a fixed-bed tubular reactor. Conventional, commercial fixed-bed ethylene-oxide reactors are typically in the form of a plurality of parallel elongated tubes (in a suitable shell) approximately 0.7 to 2.7 inches O.D. and 0.5 to 2.5 inches I.D. and 15-53 feet long filled with catalyst. Such reactors include a reactor outlet which allows the olefin oxide, un-used reactant, and byproducts to exit the reactor chamber.

Typical operating conditions for the ethylene epoxidation process involve temperatures in the range from about 180° C. to about 330° C., and preferably, from about 200° C. to about 325° C., and more preferably from about 225° C. to about 280° C. The operating pressure may vary from about atmospheric pressure to about 30 atmospheres, depending on the mass velocity and productivity desired. Higher pressures may be employed within the scope of the invention. Residence times in commercial-scale reactors are generally on the order of about 0.1 to about 5 seconds. The present catalysts are effective for this process when operated within these ranges of conditions.

The resulting ethylene oxide, which exits the reactor through the reactor outlet, is separated and recovered from the reaction products using conventional methods. For this invention, the ethylene epoxidation process may include a gas recycle wherein substantially all of the reactor effluent is readmitted to a reactor inlet after substantially or partially removing the ethylene oxide product and the byproducts including carbon dioxide. In the recycle mode, carbon dioxide concentrations in the gas inlet to the reactor may be, for example, from about 0.3 to about 5 volume percent.

The inventive catalysts have been shown to be particularly selective for oxidation of ethylene with molecular oxygen to ethylene oxide especially at high ethylene and oxygen conversion rates. The conditions for carrying out such an oxidation reaction in the presence of the catalysts of the present invention broadly comprise those described in the prior art. This applies to suitable temperatures, pressures, residence times, diluent materials, moderating agents, and recycle operations, or applying successive conversions in different reactors to increase the yields of ethylene oxide. The use of the present catalysts in ethylene oxidation reactions is in no way limited to the use of specific conditions among those which are known to be effective.

For purposes of illustration only, the following are conditions that are often used in current commercial ethylene oxide reactor units: a gas hourly space velocity (GHSV) of 1500-10,000 h⁻¹, a reactor inlet pressure of 150-400 psig, a coolant temperature of 180-315° C., an oxygen conversion level of 10-60%, and an EO production rate (work rate) of 7-20 lbs. EO/cu.ft. catalyst/hr. The feed composition at the reactor inlet may typically comprises 1-40% ethylene, 3-12% O₂, 0.3-40% CO₂, 0-3% ethane, 0.3-20 ppmv total concentration of organic chloride moderator(s), and the balance of the feed being comprised of argon, methane, nitrogen or mixtures thereof.

Examples have been provided below for the purpose of further illustrating the invention. The scope of this invention is not to be, in any way, limited by the examples set forth herein.

### EXAMPLES

### Example 1 (Comparative)

An alumina carrier was impregnated with a water-based impregnation solution containing silver in the form of silver-amine oxalate and catalytically active amounts of promoters in soluble form. The amount of silver deposited on the carrier was about 16 wt % of the carrier. After impregnation, the carrier was calcined under standard conditions.

Silver solution.

A silver solution was prepared using the following components (parts by weight):
i. Silver oxide—800 parts
ii. Oxalic acid—426.5 parts
iii. Ethylene diamine—543.6 parts
iv. Deionized water—695.5 parts
   First, deionized water was placed in a cooling bath to maintain temperature during the whole preparation under 45° C. At continuous stirring, ethylenediamine was added in small portions to avoid overheating. Oxalic acid dihydrate was then added to the water-ethylenediamine solution in small portions. After all oxalic acid was dissolved, high purity silver oxide was added to solution in small portions. After all silver oxide was dissolved and the solution was cooled to about 35° C. it was removed from the cooling bath and filtered. After filtration, the solution contained roughly 30 wt % silver, and had a specific gravity of 1.55 g/mL.

Catalyst Preparation-Impregnation.

To the above silver solution at thorough mixing promoters were added in catalytically active amounts individually or as a mixture of aqueous based solutions. For example, Cs as CsOH, Li as LiNO₃, Re as HReO₄, W as ammonium metatungstate, and S and (NH₄)₂SO₄.

A 100 g to 300 g of carrier sample was placed in a flask and then exposed to vacuum until the pressure was below 20 mm Hg. 200-300 ml of the silver/promoter solution to cover the carrier was introduced to the flask under vacuum. The vacuum was released after about 5 minutes to restore ambient pressure, hastening complete penetration of the solution into the pores. Subsequently, the excess impregnation solution was drained from the impregnated carrier.

In the catalyst composition the silver content was at nominal 16.5%. The promoters content was optimized to provide maximum stability at high selectivity. High selectivity was achieved by maintaining concentrations of Cs in the range of 400 ppm to 1000 ppm, Li in the range 100-200 ppm, Re in the range 200-400 ppm, W in the range 50-200 ppm, and S in the range 20-100 ppm on the catalyst.

Calcination.

Carrier impregnated with silver solution with promoters was calcined on a belt calciner under nitrogen atmosphere. Nitrogen flow into the calciner was optimized to remove volatile components and to protect the calciner atmosphere from contamination by outside air. Oxygen typically was kept at or below 10 ppm.

The impregnated carrier entered the calciner on a moving belt. The belt speed and the calciner oven sections temperatures were optimized to reach 400° C. as measured by a thermocouple positioned in the catalyst bed in app. 15 min Upon reaching 400° C. the catalyst was cooled in app. 20 min to 30-35° C. at which point it leaves the nitrogen atmosphere and exits the calciner.

### Example 2 (Inventive)

Example 2 was prepared in the same way as Example 1, except that after impregnation of the carrier and before calcination, the carrier was heated in an oven for 30 minutes at 80° C. After the heat treatment, the carrier was calcined under the same conditions as Example 1.

### Example 3 (Comparative)

An alumina carrier (the same carrier material as used in Examples 1 and 2) was impregnated with a water based impregnation solution containing silver in the form of silver-amine oxalate, but in the absence any amount of promoters in soluble form. After impregnation the material was calcined under conventional conditions.

### Example 4: Performance Testing of Catalysts Prepared in Examples 1 and 2

The catalysts prepared from Examples 1 and 2 were tested for their catalytic performance. The results are shown in Table 1. It is evident that the catalyst prepared with the heat treatment step (Example 2) before calcination evidenced improved selectivity, stability and activity.

TABLE 1

| Catalyst | | | | | | |
|---|---|---|---|---|---|---|
| Preparation | S_{500 h} % | S_{1500 h} % | S_{3000 h} % | T_{500 h} ° C. | T_{1500 h} ° C. | T_{300 h} ° C. |
| Example 1 w/o Heat | 89.3 | 89.2 | 87.6 | 248 | 255.6 | 262.7 |
| Treatment Step | | | | | | |
| Example 2 with Heat | 90.3 | 89.9 | 89.7 | 243 | 251.6 | 260 |
| Treatment Step | | | | | | |

### Example 5: XPS Analysis of Catalysts Prepared in Examples 1, 2 and 3

The catalysts prepared according to Examples 1, 2 and 3 were analyzed by x-ray photoelectron spectroscopy (XPS) to determine the near surface silver concentration in atomic %. The results are shown in Table 2.

TABLE 2

| | Ag in Catalyst | | Loss |
|---|---|---|---|
| Catalyst Preparation | Composition % | Ag Atomic % | of Ag Signal % |
| Example 1 | 16.3 | 20.5 | 8.4 |
| Example 2 | 16.3 | 11.4 | 38.7 |
| Example 3 | 15.74 | 22.50 | |

Table 2 above, shows the silver concentration near the surface of the catalyst in atomic % from XPS analysis of the catalyst prepared by conventional impregnation with soluble promoters and without a heat treatment step (Example 1), the catalyst prepared by conventional impregnation with soluble promoters and with a heat treatment step (Example 2), and the catalyst prepared by conventional impregnation with no soluble promoters and no heat treatment step.

The results in Table 2 demonstrate that the near surface silver concentration decreases more than 20% after the inventive heat treatment step.

As the XPS silver signal diminishes with increasing coverage by promoters, the results in Table 2 indicate that the heat treatment step prior to calcination results in greater coverage of the overall total silver by promoters than the catalyst prepared without the heat treatment step. Thus, the catalyst prepared according to Example 2 (inventive) exhibits an improved selectivity, activity and stability compared to the catalyst prepared by conventional processes (Example 1). The results suggest the improved performance is due to the silver coverage by the promoters, at least in part, and in the creation of more active sites on the catalyst surface.

What is claimed is:

1. A method for the preparation of a silver-based catalyst effective for the conversion of ethylene to ethylene oxide, the method comprising:
co-impregnating a porous refractory carrier with a solution comprising a catalytically effective amount of silver and a promoting amount of rhenium, wherein after said co-impregnation is complete, the co-impregnated carrier is heated at a temperature of about 40° C. to about 300° C. for a duration of about 1 minute to about 120 minutes; and thereafter calcining the co-impregnated carrier for a time and at a temperature sufficient to convert the silver to an active species.

2. The method of claim 1 wherein the co-impregnated carrier is heated for about 10 minutes to about 60 minutes at a temperature between about 50° C. and about 200° C.

3. The method of claim 2 wherein the co-impregnated carrier is heated for about 20 minutes to about 30 minutes at a temperature between about 60° C. and about 100° C.

4. The method of claim 1 wherein the co-impregnated carrier is heated at about 80° C. for about 30 minutes.

5. The method of claim 1 wherein said carrier is co-impregnated with a catalytically effective amount of a promoter selected from the group consisting of alkali metals.

6. The method of claim 1 wherein said carrier is co-impregnated with a catalytically effective amount of a promoter selected from the group consisting of alkaline earth metals of Group IIA of the Periodic Table.

7. The method of claim 1 wherein said carrier is co-impregnated with a catalytically effective amount of a promoter selected from the group consisting of transition metals from Groups IVA, VA, VIA, VIIA and VIIIA of the Periodic Table.

8. The method of claim 1 wherein the carrier is an α-alumina carrier.

9. A silver-based epoxidation catalyst comprising a catalyst surface wherein the Ag on the surface of the catalyst is covered by more than 30% with promoters as measured by a loss of the surface atomic concentration of Ag is XPS analysis.
